(19) United States
(12) Patent Application Publication
(10) Pub. No.: US 2002/0077352 A1
(43) Pub. Date: Jun. 20, 2002

(54) N-methyl-D-aspartate receptor antagonists

(76) Inventors: Nikolaus J. Sucher, Kowloon (HK); Xiandi Gong, New Territories (CA); Maria Carles, Kowloon (HK); Nancy Ip, Hong Kong (HK); Feng Chen, Hong Kong (HK)

Correspondence Address: BAKER & McKENZIE, 12th Floor, 101 West Broadway, San Diego, CA, 92101, US

(21) Appl. No.: 09/922,535

(22) Filed: Aug. 3, 2001

Related U.S. Application Data
(60) Provisional application No. 60/222,802, filed on Aug. 3, 2000.

(51) Int. Cl. A61K 31/343; A61K 31/122; C07D 307/93

(52) U.S. Cl. 514/468; 514/680; 552/292; 549/457

(57) ABSTRACT

The present invention is based in part on the surprising discovery that Tanshinones from Salvia miltiorrhiza act as allosteric high-potency N-methyl-D-aspartate receptor antagonists. Pharmacological blockade of excessive activation of N-methyl-D-aspartate receptors (NMDARs) greatly reduces ischemic injury of neurons in cell culture and animal models. Tanshinones thus represent a novel class of compounds with NMDA receptor blocking activities with potential for the development of safe neuroprotective drugs for therapy of stroke and other neurodegenerative and neuropsychiatric disorders.

### CROSS-REFERENCE TO RELATED APPLICATIONS

The present application claims priority to U.S. Provisional Application No. 60/222,802, filed Aug. 3, 2000 and entitled “Novel Allosteric High-Affinity N-methyl-D-Aspartate Receptor Antagonists”, which is incorporated herein by reference in its entirety, including any drawings.

### FIELD OF THE INVENTION

The present invention relates to the field of N-methyl-D-aspartate receptor antagonists. More particularly, the present invention relates to novel uses for particular traditional Chinese medicines and related compounds and compositions.

### BACKGROUND OF THE INVENTION

A large body of experimental evidence indicates that excessive activation of N-methyl-D-aspartate receptors (NMDARs) mediates the calcium-dependent neurotoxicity associated with ischemic, hypoglycemic and degenerative injury of neurons (Lipton, S. A. and P. A. Rosenberg, 1994, New England Journal of Medicine 330, 613; Meldrum, B. S., 1992, Curr Opin Neurol Neurosurg 5, 508; and Meldrum, B. S., et al. 1987, Med Biol 65, 153). NMDARs play a major role in physiological processes such as neuronal development, synaptic plasticity, and generation of long-lasting memory in the central nervous system.

NMDAR antagonist drugs are commonly classified according to their primary site of action as 1) competitive inhibitors acting at the NMDA recognition site, 2) competitive inhibitors acting at the binding site of the co-agonist glycine, 3) channel blockers or 4) allosteric modulators (for a review see Sucher, N. J., M. Awobuluyi, Y. B. Choi and S. A. Lipton, 1996, Trends in Pharmacological Sciences 17, 348).

Phenylethanolamines (exemplified by ifenprodil) are allosteric NMDAR inhibitors that inhibit NMDARs by enhancing proton inhibition (Mott et al., 1998; Nature Neuroscience 1(8):659-667). A hallmark of this group of NMDAR antagonists is their selectivity for NMDARs containing the NR2B subunit with virtual loss of blocking activity in receptors containing the NR2D subunit (rev. in ref. Sucher et al., 1996; Trends in Pharmacological Sciences 17, 348).

Although pharmacological blockade of NMDARs has been shown to protect neurons from insults in cell culture and animal models, no NMDAR antagonist has proven to date to be effective and safe in humans (Goldzmidt, A. and R. J. Wityk, 1998, Current Opinion in Neurology 11, 57; Li, L. N., 1998, Pure & Applied Chemistry 70, 547).

Clinical experience in China indicated that traditional Chinese medicines (TCMs) prevented or abated disability in stroke patients (Chen, K., 1995, Trends in Pharmacological Sciences 16, 182; Gong, X. and N. J. Sucher, 1999, Trends in Pharmacological Sciences 20, 191; Zhou, S. and P. Xiao, 1997, A modern practical handbook of neurology and psychosis of the integration of traditional Chinese and Western medicine (Hunan Science and Technology Publisher P. R. China)).

One TCM of particular interest in this context is the root of S. miltiorrhiza named Danshen or Tan-Shen in Chinese. Danshen is an ancient Chinese drug, and more recently it has been determined that the red color imparts to its roots is due to the accumulation of lipid-soluble diterpenoid quinones named tanshinones, one group of bioactive secondary metabolites of the plant.

Despite the existing knowledge regarding TCMs and NMDAR antagonists, there remains a need for the identification of additional NMDAR antagonists as well as a need to expand our understanding of the chemical diversity inherent in TCMs. In the process, it is envisioned that researchers will discover and develop evermore novel and effective NMDR therapeutics and research tool, in addition to identifying other non-NMDAR uses for active components thereof.

### SUMMARY OF THE INVENTION

Based on the described NMDAR antagonist properties of tanshinones, therapeutic applications would encompass all those in which excessive or damaging activity of NMDARs have been implicated. Such disorders include ischemic (stroke), epileptic, neurodegenerative (Parkinson's disease, Huntington's disease, amyotrophic lateral sclerosis, Alzheimer's disease etc.) and neuropsychiatric diseases (addiction, chronic pain). It is noteworthy that tanshinones also possess anti-inflammatory and antioxidant properties, which are likely of additional benefit in these therapeutic applications.

Tanshinones have a therapeutic advantage in that they inhibit effects of excess glutamate in affected areas of the brain, with less influence on normal receptor function. In addition to being high-affinity NMDAR blockers, tanshinones may afford additional benefits in the treatment of cerebral ischemia and other NMDAR-mediated neurodegenerative and neuropsychiatric diseases.

A subgroup of tanshinones, represented by miltirone, has also been described as partial agonists at central benzodiazepine receptors and the dual property of NMDAR antagonist and partial benzodiazepine agonist may be of therapeutic advantage in some applications.

Thus, in one aspect, the present invention provides a substantially purified compound having structure I, wherein R₁ and R₂ are independently selected from the group consisting of H, OH and CH₃ and R₃ is selected from the group consisting of H and CH₃.

By “substantially purified” is meant that the compound is relatively purer than it is in the natural environment. Preferably, the compound is at least 50%, 75%, 90%, 95%, 98%, 99% or 100% pure as measured by weight or volume.

In another aspect, the invention features a pharmaceutical or standardized composition comprising one or more compounds having structure I, wherein the substituents are as defined above.

In yet another aspect, the present invention provides a method of preventing or treating a disease or condition. The method involves the step of administering a compound or composition of the invention to a patent in need of such treatment.

In another aspect, the present invention provides a method of using a compound having structure I (with the substituents defined as above) as biological, functional and/or physical markers for establishing safety and standardization protocols for dietary food supplements or complex drugs.

The present invention also features a method of manufacturing a pharmaceutical composition including the step of extracting a compound having structure I (with the substituents defined as above) or utilizing a compound having structure I (with the substituents defined as above) in its substantially pure form.

In preferred embodiments, the compound is tanshinone, cryptotanshinone (structure III), miltirone (structure IV), tanshinone I (structure V), tanshinone IIA (structure VI), tanshinone IIB (structure VII), or a derivative or prodrug thereof. In one embodiment the composition includes about 2.13% miltirone I, 7.35% cryptotanshinone, 3.24% tanshinone I and 6.72% tanshinone IIA by weight in a 0.35g capsule (0.26 g powder and 0.09 g coat). The disease or condition preferably is: (1) a neurological disease and/or condition, in particular neurological diseases and/or conditions associated with the NMDA-receptor; (2) stroke, such as a global ischemic, hemorrhagic, or focal ischemic stroke; (3) chronic pain; (4) acute neurological trauma; (5) general dementia, such as Alzheimer's disease, AIDS-related dementia, age-related dementia, or multi-infarct dementia; (6) glaucoma; or (7) tolerance, sensitization and drug addiction preferably (provided the compound is not tanshinone IIA).

While certain aspects of the invention have been summarized herein, other useful applications, embodiments, and aspects of the invention are disclosed in the detailed description and in the drawings.

### BRIEF DESCRIPTION OF THE DRAWINGS

These and other features and advantages of the present invention will be appreciated from the following detailed description, along with the accompanying figures in which like reference numerals identify like elements throughout and wherein:

FIG. 1(a) shows blockade of NMDARs in cultured cortical neurons by aqueous extracts of Salvia miltiorrhiza roots and tanshinones. NMDA (200 μM) alone, NMDA plus aqueous Salvia miltiorrhiza extract (100 μl in 10 ml Hanks solution) and NMDA plus 20 nM tanshinone II_{A} were applied for about 3 seconds (black bars) in the continuous presence of glycine (10 μM) and absence of Mg²⁺.

FIG. 1(b) shows HPLC chromatograms of aqueous extracts from Salvia miltiorrhiza roots (top) and HPLC chromatogram showing eight standard compounds (bottom; see Table 1).

FIG. 1(c) shows chemical structures of tanshinones.

FIG. 1(d) shows block of NMDA-induced responses in cultured cortical neurons by tanshinone I, tanshinone II_{A}, tanshinone II_{B}, miltirone, and cryptotanshinone (20 nM).

FIG. 2(a) shows inhibition of NMDA-evoked currents by tanshinones wherein NMDA (200 μM) or NMDA plus increasing concentrations of tanshinone II_{A} (0.002 nM-20 nM) were applied for about 3 seconds (black bars) in the continuous presence of glycine (10 μM) and absence of Mg²⁺.

FIG. 2(b) shows inhibition of NMDA-evoked currents by tanshinones wherein the concentration-dependent inhibition of NMDA-evoked currents by tanshinone IIB (empty square), cryptotanshinone (empty circle), miltirone (filled diamond), tanshinone II_{A} (curve only for clarity), and tanshinone I (empty triangle). The ordinate indicates the percent steady-state block of currents evoked by 200 μM NMDA plus 10 μM glycine in the absence of Mg²⁺. Each point represents the mean value from three or four cells; error bars indicate S.E.M. (see text for calculated IC₅₀ values and 95% confidence intervals).

FIG. 3(a) shows non-competitive inhibition of NMDA-activated responses by tanshinone II_{A} in cultured cortical neurons. NMDA (100 μM) or increasing concentrations of NMDA (100 μM-1600 μM) plus tanshinone II_{A} (20 nM) were applied for about 3 seconds (indicated by the black bars) in the continuous presence of glycine (10 μM) and absence of Mg²⁺,

FIG. 3(b) shows representative current/voltage (I/V) plots of NMDA-evoked responses in cultured corticalneurons in the presence of tanshinone I, tanshinone H_{A}, tanshinone II_{B}, miltirone, or cryptotanshinone. I/V curves were constructed by subtracting the response in the presence of NMDA (200 μM) plus tanshinone from the control response during the application of NMDA alone for 2 seconds at membrane holding potentials from −60 mV to +60 mV. Data from 2 to 5 sweeps were averaged for each trace.

FIG. 3(c) shows effect of tanshinone IIA and IIB on recombinant NMDARs composed of NR1 and NR2D subunits in HEK293 cells.

FIG. 4 shows structure I.

FIGS. 5 and 6 show generic structures.

Some or all of the Figures may be schematic representations for purposes of illustration and do not necessarily depict the actual relative sizes or locations of the elements shown.

### DETAILED DESCRIPTION OF THE INVENTION

In the following paragraphs, the present invention will be described in detail by way of example with reference to the figures. Throughout this description, the preferred embodiment and examples shown should not be considered as limiting the scope of the present invention.

The whole-cell patch clamp technique was used in mouse cortical neuronal cultures in order to screen for NMDAR antagonists in Danshen, the root of Salvia milthiorriza Bunge. Danshen water extracts contained high potency, non-competitive NMDAR antagonists with a readily reversible mode of action. The NMDAR antagonists were identified as tanshinones. Tanshinones are plant-derived diterpenoid quinones.

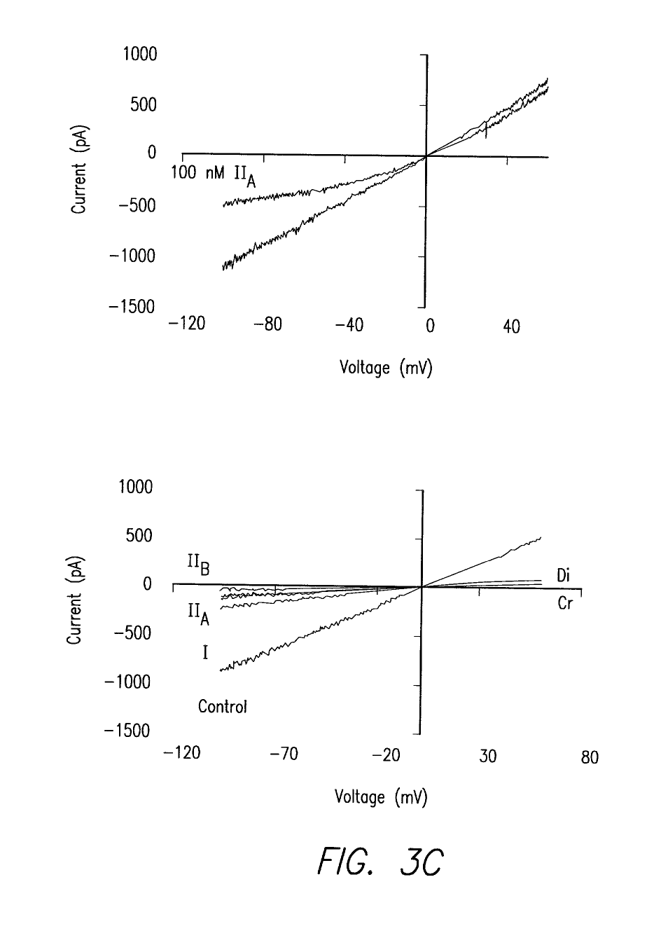

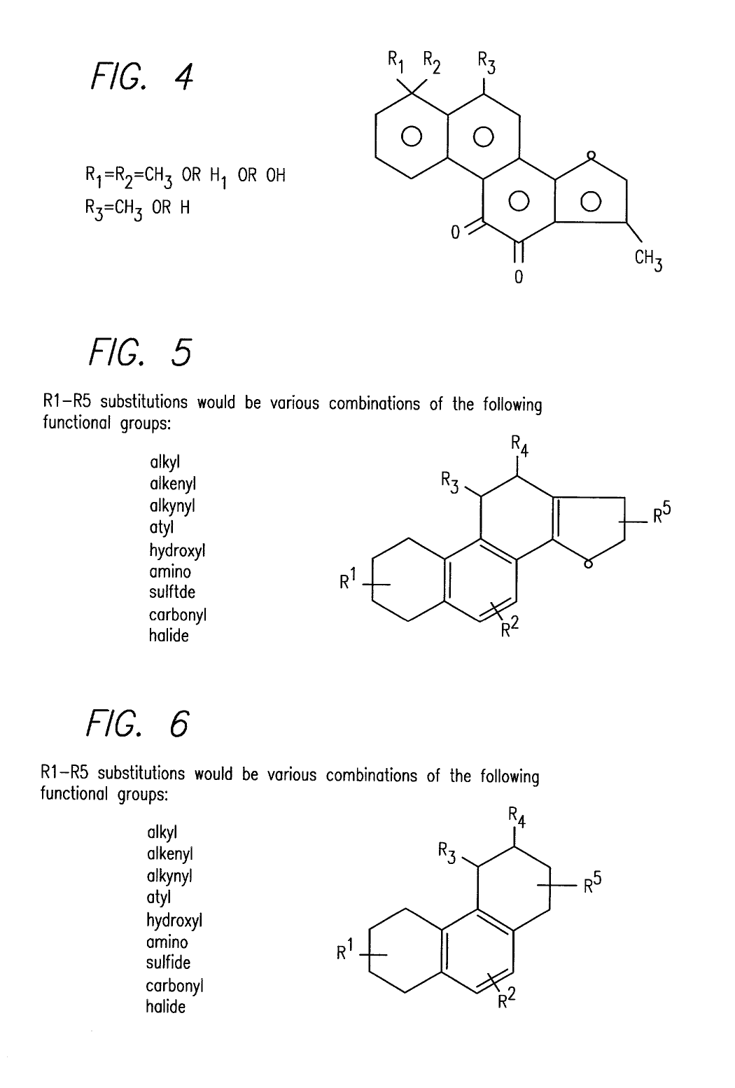

Compounds of the generic structures shown in FIGS. 5 and 6 are also expected to work in the present inventory and can be tested using various assays described herein or known in the art. Chemical definitions of terms used in FIGS. 5 and 6 are defined below.

As used herein, the term “alkyl” denotes branched or unbranched hydrocarbon chains containing between one and six, preferably one and four, carbon atoms, such as, e.g., methyl, ethyl, n-propyl, iso-propyl, n-butyl, sec-butyl, iso-butyl, tert-butyl, and 2-methylpentyl. These groups may be optionally substituted with one or more functional groups which are attached commonly to such chains, such as, e.g., hydroxyl, bromo, fluoro, chloro, iodo, mercapto or thio, cyano, alkylthio, heterocycle, aryl, heteroaryl, carboxyl, alkoxycarbonyl, alkyl, alkenyl, nitro, amino, alkoxyl, amido, and optionally substituted isothioureido, amidino, guanidino, and the like to form alkyl groups such as trifluoromethyl, 3-hydroxyhexyl, 2-carboxypropyl, 2-fluoroethyl, carboxymethyl, 4-cyanobutyl, 2-guanidinoethyl, 3-N,N′-dimethylisothiouroniumpropyl, and the like.

The term “alkenyl” denotes an alkyl group as defined above having at least one double bond, e.g., allyl, 3-hydroxy-2-buten-1-yl, 1-methyl-2-propen-1-yl and the like.

The term “alkynyl” denotes an alkyl group as defined above having at least one triple bond.

The term “aryl” denotes a chain of carbon atoms an which form an least one aromatic ring having preferably between about 6-14 carbon atoms, such as, e.g., phenyl, naphthyl, indenyl, and the like, and which may be substituted with one or more functional groups which are attached commonly to such chains, such as, e.g., hydroxyl, bromo, fluoro, chloro, iodo, mercapto or thio, cyano, cyanoamido, alkylthio, heterocycle, aryl, heteroaryl, carboxyl, alkoxycarbonyl, alkyl, alkenyl, nitro, amino, alkoxyl, amido, and the like to form aryl groups such as biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolylmethylphenyl, cyanophenyl, pyridylphenyl, pyrrolylphenyl, pyrazolylphenyl, triazolylphenyl, tetrazolylphenyl and the like.

The term “hydroxyl” denotes an OH group.

The term “amino” denotes the group NRR′, where R and R′ may independently be alkyl, aryl or acyl as defined above, or hydrogen.

The term “sulfide” denotes a sulfur or S group.

The term “carbonyl” denotes compounds with a carbon to oxygen double bond (C═O), including aldehydes and ketones.

The term “halide” denotes a binary combination of a halogen with another element, such as potassium iodide KI, or an organic compound in which halogen atoms replace one or more hydrogen atoms, for example CH₃Cl.

Such compounds can be tested in various assays including:

| Description | Cite |
|---|---|
| Ischemic damage | Koroshetz W. J., Moskowitz M. A. Emerging treatments for |
| | stroke in humans. TiPS 1996; 17: 227-233. |
| Animal models | Hunter A. J., Mackay K. B., Rogers D. C. To what extent have |
| | functional studies of ischemia in animals been useful in the |
| | assessment of potential neuroprotective agents. TiPS 1999; |
| | 19: 59-66. |
| Neurodegeneration | Lipton S. A., Rosenberg P. A. Excitatory amino acids as a final |
| | common pathway for neurologic disorders. N. Engl. J. Med. 1994 |
| | Mar 3; 330(9): 613-22. |
| Glaucoma and AIDS | Lipton S. A. Retinal ganglion cells, glaucoma and |
| related dementia | neuroprotection. Prog. Brain Res. 2001; 131: 712-8. |
| | Kaul M., Garden G. A., Lipton S. A. Pathways to neuronal injury |
| | and apoptosis in HIV-associated dementia. Nature 2001 Apr 19; 410. |
| | Lipton S. A. Neuronal injury associated with HIV-1: approaches |
| | to treatment. Annu. Rev. Pharmacol. Todicol. |
| General | Nicotera P., Lipton S. A. Excitotoxins in neuronal apoptosis and |
| | necrosis. J. Cereb. Blood Flow Metab. 1999 Jun; 19(6): 583-91. |
| | Jonas S., Ayigari V., Viera D., Waterman P. Neuroprotection |
| | against cerebral ischemia. A review of animal studies and |
| | correlation with human trial results. Ann. N. Y. Acad. Sci. 1999; |
| | 890: 2-3. |
| Grand mal epilepsy | Toman J. E. P. Animal techniques for evaluating anticonvulsants. |
| | In: Nodin J. H. and Siegler P. E. (eds) Animals and clinical |
| | Techniques in Drug Evaluation. year Book Med. Publ. 1964, |
| | vol. 1: 348-352. |
| Systemic convulsant | Leander J. D., Lawson R. R., Ornstein P. L., Zimmerman D. M. N- |
| models | methyl-D-aspartate acid induced lethality in mice: selective |
| | antagonism by phencyclidine-like drugs. Brain Res. (1988) |
| | 448: 115-120. |
| | Pollack G. M., Shen D. D. A timed intravenous pentylenetetrazol |
| | infusion seizure model for quantitating the anticonvulsant effect |
| | of valproic acid in the rat. J. Pharmacol. Meth. (1985) 13: 135-146. |
| | Snead III O. C. γ-Hydroxybutyrate model of generalized absence |
| | seizures: Further characterization and comparison with other |
| | absence models. Epilepsia (1988) 29: 361-368. |
| | Stone W. E. Systemic chemical convulsants and metabolic |
| | derangement. In: Purpura D. P., Penry J. K., Tower D. B., |
| | Woodbury D. M., Walter R. D. (eds) Experimental Models of |
| | Epilepsy: A manual for the Laboratory Worker. Raven Press, |
| | New York (1972) pp. 407-432. |
| Kindled rat seizure | Girgis M. Kindling as a model for limbic epilepsy. Neurosci. |
| | (1981) 6: 1695-1706. |
| | Pinel J. P. J., Rovner L. I. Experimental epileptogenesis: kindling- |
| | induced epilepsy in rats. Exper. Neurol. (1978) 58: 190-202. |
| Genetic animal | Löscher, W. Genetic animal models of epilepsy as a unique |
| models of epilepsy | resource for the evaluation of anticonvulsant drugs. A review. |
| | Meth. Fing. Exptl. Clin. Pharmacol. (1984) 6: 531-547. |
| | Oguro K., Ito M., Tsuda H., Mutoh K., Shiraishi H., Shirasaka |
| | Y., Mikawa H. Associated of NMDA receptor sites and seizures |
| | E1 mice. Epilepsy Res. (1991) 9: 255-230. |
| | Seyfried T. N. Audiogenic seizures in mice. Fed. Proc. (1979) |
| | 38: 2399-2404. |
| DBA/2 mouse | Carling R. W., Leeson P. D., Moore K. W., Smith J. D., Moyes |
| anticonvulsant | C. R., Mawer I. M., Thomas S., Chan T., Baker R., Foster A. C. 3- |
| | Nitro-3, 4-dihydro-2 (1H)-quinolones. Excitatory amino acid |
| | antagonists acting at glycine-site NMDA and (RS)-alpha-amino- |
| | 3-hydroxy-5methyl-4-isoxazolepropionic acid receptors. J. Med. |
| | Chem. (1993) 36(22): 3397-408. |
| Neuropathic pain | Inoue T., Mashimo T., Shibata M., Shibuta S., Yoshiya I. Radip |
| | development of nitric oxide-induced hyperalgesia depends on an |
| | alternate to the cGMP-mediated pathway in the rat neuropathic |
| | pain model. Brain Res. (1998) 792(2): 263-70. |
| | Stevens C. W. An amphibian model for pain research. Lab |
| | Animal (1995) 24: 32-36. |
| | Stevens C. W. Alternatives to the use of mammals for pain |
| | research. Life Sciences (1992) 50: 901-912. |
| | Kavaliers M. K., Ossenkopp K. P., Sanberg P. R. (eds), Animal |
| | Models of Nociception and Pain (1997) R. G. Landes Co.: |
| | Austin. |

The particular compound that affects the disorder of interest can be administered to a patient either by themselves, or in pharmaceutical compositions where it is mixed with suitable carriers or excipient(s). In treating a patient exhibiting a disorder of interest, a therapeutically effective amount of a agent or agents such as these is administered. A therapeutically effective dose refers to that amount of the compound that results in amelioration of symptoms or a prolongation of survival in a patient.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays. For example, a dose can be formulated in animal models to achieve a circulating plasma concentration range that includes the IC₅₀ as determined in cell culture (i.e., the concentration of the test compound which achieves a half-maximal disruption of the protein complex, or a half-maximal inhibition of the cellular level and/or activity of a complex component). Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the oncogenic disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few. For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve a 50-90% inhibition of the kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90%. 578902vl

### EXAMPLES

Several preferred embodiments in accordance with the invention are described using the following examples for illustration:

### Example 1

### Blockade Of NMDARS In Cultured Cortical Neurons By Tanshinones

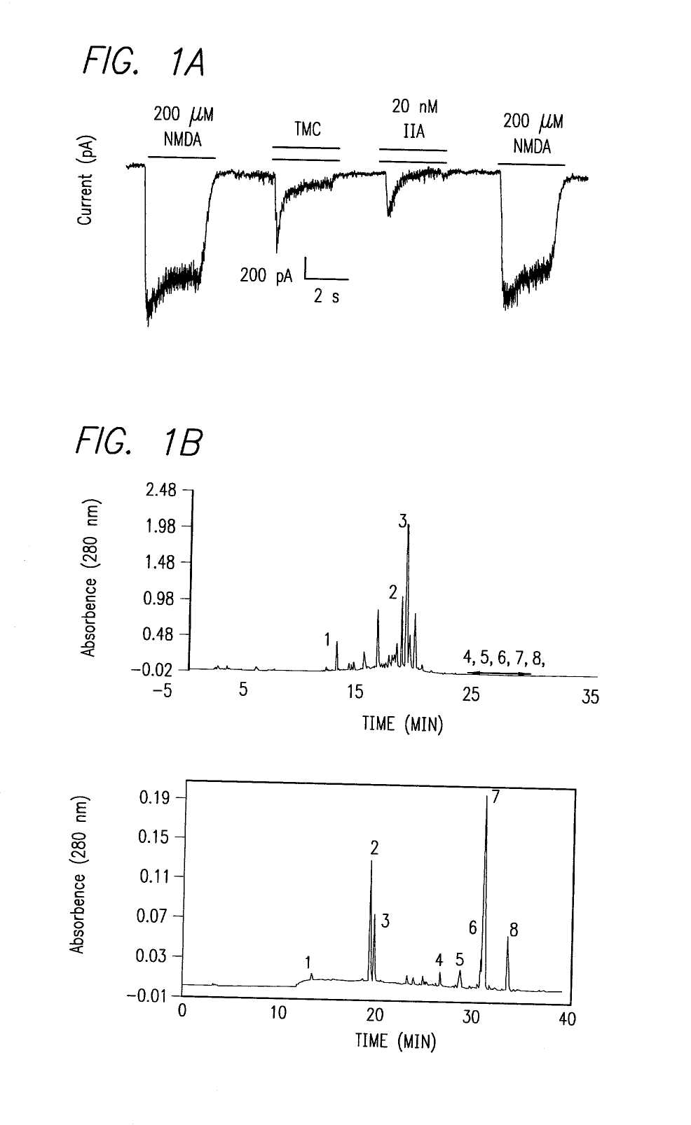

NMDA-evoked currents in cultured mouse cortical neurons were blocked effectively in the presence of 1% (v/v) aqueous Danshen extract (FIG. 1a). The major components in aqueous extracts of the Salvia miltiorrhiza roots are water-soluble compounds such as tanshinol, rosmarinic acid, and salvianolic acid (Table 1; FIG. 1b).

However, none of these major chemical components of the aqueous extracts showed NMDAR antagonist activity even at millimolar concentration. Aqueous Danshen extracts contain only very small amounts of tanshinones. In order to investigate whether tanshinones were the effective components in the water extracts, a series of purified tanshinones were tested in the patch-clamp assay. Cryptotanshinone, miltirone, tanshinone I, tanshinone IIA, and tanshinone IIB produced a strong, but readily reversible inhibition of the NMDA-induced currents similar to the effect of the aqueous extracts (FIGS. 1c, d).

TABLE 1
Concentrations of pharmacologically active components
in aqueous extracts of Salvia miltiorrhiza roots

| | Retention | | |
|---|---|---|---|
| Peak No. | time (min) | Compound | Concentration (μM) |
| 1 | 12.61 | tanshinol | 964.61 ± 45.86 |
| 2 | 18.21 | rosmarinic acid | 553.45 ± 57.68 |
| 3 | 18.6 | salvianolicacid | 2542.71 ± 134.91 |
| 4 | 25.08 | tanshinone II_{B} | 14.46 ± 4.32 |
| 5 | 27 | miltirone | 9.43 ± 1.02 |
| 6 | 29.04 | cryptotanshinone | 6.51 ± 0.83 |
| 7 | 29.25 | tanshinone I | 0.79 ± 0.02 |
| 8 | 31.65 | tanshinone II_{A} | 0.16 ± 0.02 |

### Example 2

### Inhibition Of NMDA-Evoked Currents By Tanshinones

The present example examined whether tanshinones inhibited currents of AMPA and GABA_{A} receptors evoked by AMPA or GABA, respectively. Currents activated by AMPA (10 μM) in mouse cultured cortical neurons showed either no, or only a small, inhibition by tanshinones (6±3% at 100 nM tanshinone IIA). Similarly, currents activated by GABA (20 μM) were blocked by 10±3% (n=3).

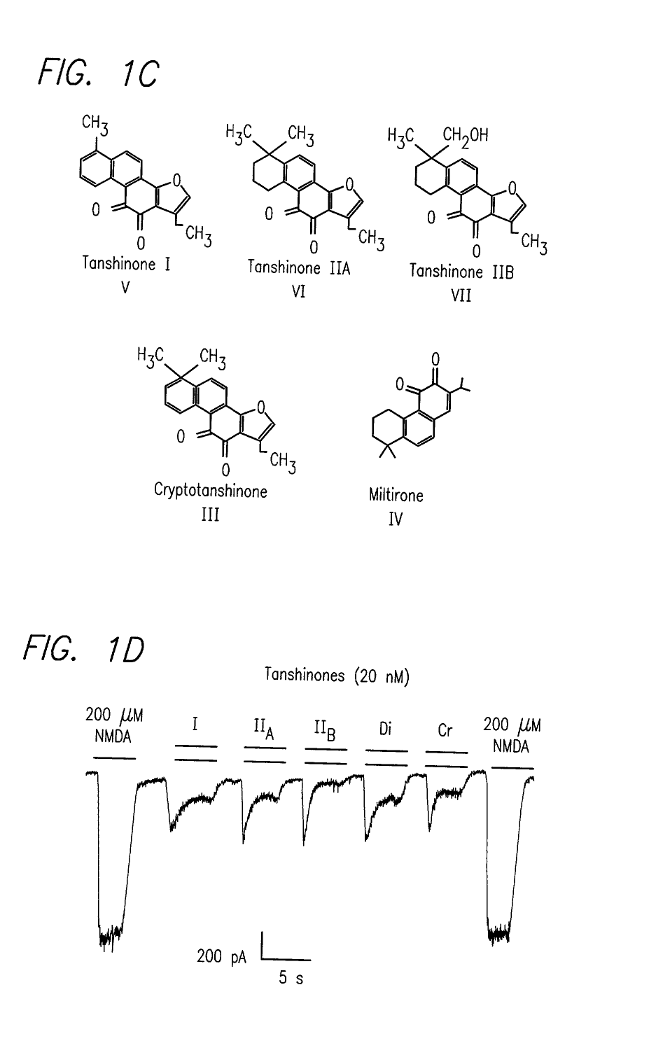

The concentration dependence of the steady-state block of NMDA-induced currents was determined in order to obtain the dose/response curve of the NMDAR antagonist action of the tanshinones (FIG. 2). The data were fit according to a logistic equation and the concentration at which 50% of the NMDA-evoked response was blocked (IC₅₀) was calculated from the equation. The IC₅₀ values (95% confidence interval) were 1.1 nM (0.5-1.6 nM) for tanshinone IIB, 1.7 nM (1-2.4 nM) for cryptotanshinone, 2.1 nM (1.5-2.6 nM) for miltirone, 3.4 nM (2.6-4.2 nM) for tanshinone II_{A} and 3.7 nM (2.7-4.7 nM) for tanshinone I. The electrophysiological determined IC50 values of the tanshinones are up to two orders of magnitude smaller than those of other NMDAR antagonists.

### Example 3

### Voltage-Dependence of The Inhibitory Effects of The Tanshinones

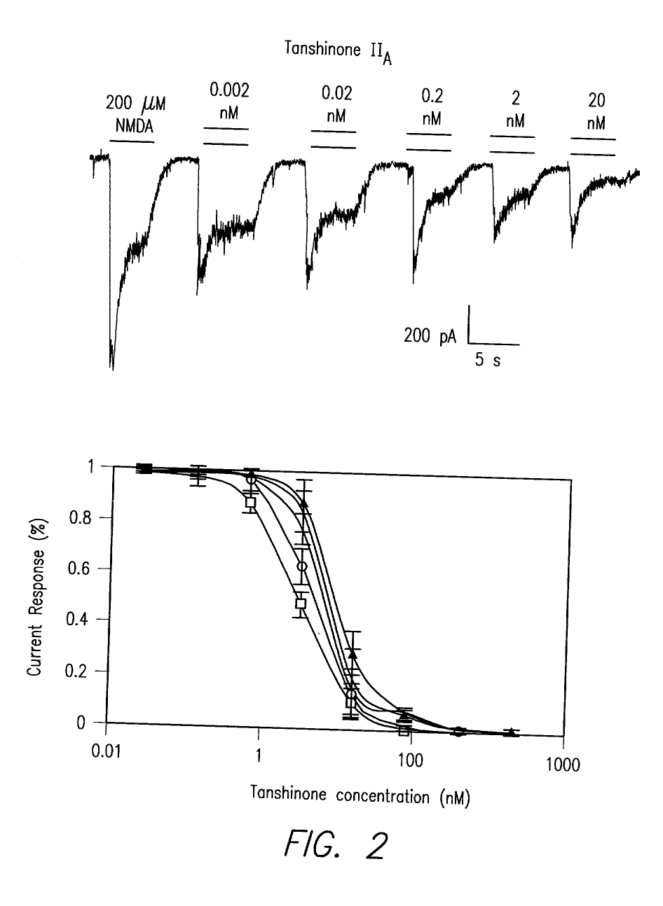

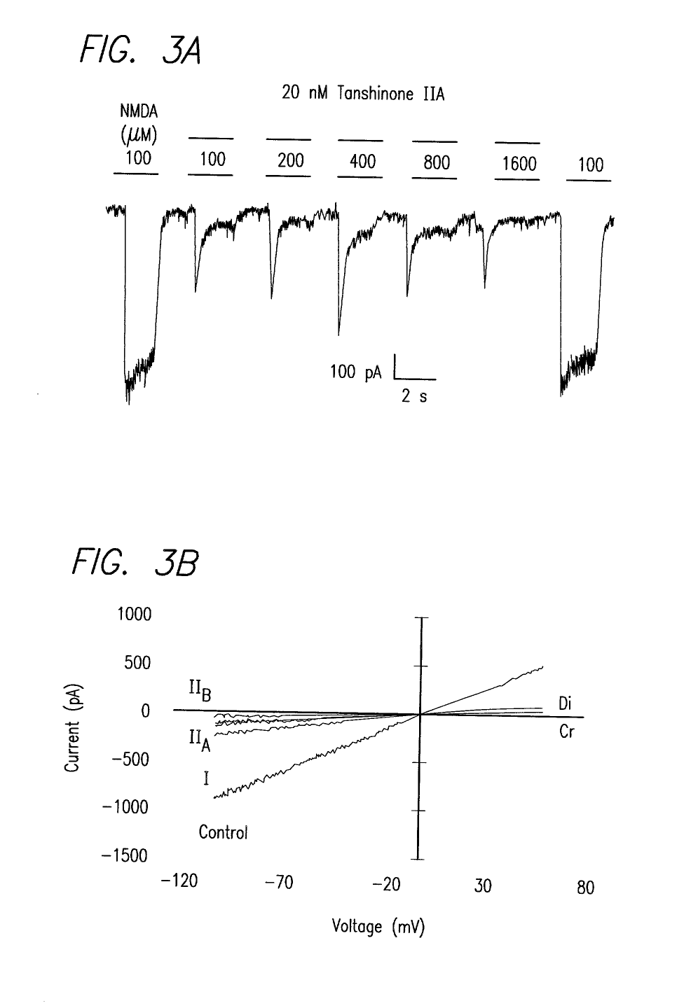

Concentration-response curves were obtained in the presence of a saturating dose of tanshinone and increasing amounts of NMDA in cultured mouse cortical neurons (FIG. 3a). The data indicated that the degree of inhibition of the NMDA-evoked current by a fixed dose of tanshinone was independent of the dose of co-applied agonist. Similarly, NMDAR inhibition by tanshinones was not affected by increasing concentrations of co-agonist glycine. Thus, tanshinones act as non-competitive blockers of NMDARs. Consistent with the lack of competition with NMDA and glycine, no whole-cell currents were observed when Danshen water extracts were either co-applied with NMDA (200 μM) in the absence of glycine or with glycine in the absence of NMDA. These data indicate that it is unlikely that tanshinones act as partial agonists at the glutamate or glycine recognition sites.

The voltage-dependence of the inhibitory effects of the tanshinones was assessed by constructing current/voltage (I/V) plots in the presence or absence of the drugs (FIG. 3b). The reversal potential of NMDA-induced currents was close to 0 mV. Similar to the effect of the water extract, the inhibition of the NMDA-induced whole-cell currents by cryptotanshinone, miltirone, and tanshinones II_{A}, II_{B}, and I was voltage-independent. Tanshinones had no discernible effect on NMDA-evoked currents when they were added to the patch-pipette internal solution. The lack of voltage-dependence of inhibition makes it unlikely that the tanshinones act as channel blockers.

This example also compared the blocking efficacy of the tanshinones in transiently transfected human embryonic kidney (HEK) 293 cells expressing recombinant NMDARs containing either the NR1/NR2B or NR1/NR2D subunit combinations. Addition of Danshen water extract (1% v/v) or purified tanshinones (20 nM) reversibly inhibited approximately 80-90% of the NMDA-induced current in the absence of TCM in recombinant NMDARs composed of the NR1 and NR2B subunits. In contrast, only little blocking activity was observed in NMDARs composed of the NR1/NR2D subunits upon co-application of NMDA and glycine in the absence of Mg²⁺ with either cryptotanshinone (100 nM; 10±1%, n=3), miltirone (100 nM; 11±2%, n=3), or tanshinone I (100 nM; 4±1%, n=3). Even concentrations that were two orders of magnitude higher than the IC₅₀ of these compounds in NR2B containing receptors did not significantly block NR2D containing receptors. However, tanshinones II_{A} (100 nM) and II_{B} (100 nM) blocked 61±11% and 52±11% of the NMDA-induced current in NR2D containing NMDARs (FIG. 3c). The inhibition produced by tanshinone IIA in recombinant receptors containing the NR2D subunit was voltage-dependent with greatly reduced efficacy at positive potentials (FIG. 3c). This is in contrast to the voltage-independent inhibitory effect of the drug in recombinant NMDARs containing the NR2B subunit suggesting a subunit specific molecular blocking mechanism of the drug in the two types of NMDARs.

### CONCLUSION

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments and are exemplary and are not intended as limitations on the scope of the invention. Changes therein and other uses will occur to those skilled in the art which are encompassed within the spirit of the invention.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

One skilled in the art will appreciate that the present invention can be practiced by other than the preferred embodiments which are presented in this description for purposes of illustration and not of limitation, and the present invention is limited only by the claims that follow. It is noted that equivalents for the particular embodiments discussed in this description are also within the scope of the present invention.

What is claimed is:

1. A substantially purified compound having structure I, wherein R₁ and R₂ are independently selected from the group consisting of H, OH and CH₃ and R₃ is selected from the group consisting of H and CH₃.

2. The compound of claim 1, wherein said compound is tanshinone.

3. The compound of claim 1, wherein said compound is crytotanshinone having structure Ill.

4. The compound of claim 1, wherein said compound is miltirone having structure IV.

5. The compound of claim 1, wherein said compound is tanshinone I having structure V.

6. The compound of claim 1, wherein said compound is tanshinone IIA having structure VI.

7. The compound of claim 1, wherein said compound is tanshinone IIB having structure VII.

8. A pharmaceutical or standardized composition comprising one or more compounds having structure I, wherein R₁ and R₂ are independently selected from the group consisting of H, OH and CH₃ and R₃ is selected from the group consisting of H and CH₃.

9. The composition of claim 8, wherein said composition is a pharmaceutical composition.

10. The composition of claim 8, wherein said composition is a standardized composition.

11. The composition of claim 8, wherein said composition comprises more than one compound having structure I.

12. The composition of claim 8, wherein at least one of said compounds is tanshinone.

13. The composition of claim 8, wherein at least one of said compounds is crytotanshinone having structure III.

14. The composition of claim 8, wherein at least one of said compounds is miltirone having structure IV.

15. The composition of claim 8, wherein at least one of said compounds is tanshinone I having structure V.

16. The composition of claim 8, wherein at least one of said compounds is tanshinone IIA having structure VI.

17. The composition of claim 8, wherein at least one of said compounds is tanshinone IIB having structure VII.

18. A method of treating or preventing a disease or condition comprising the step of providing a compound having structure I, wherein R₁ and R₂ are independently selected from the group consisting of H, OH and CH₃ and R₃ is selected from the group consisting of H and CH₃ to a patient in need of such treatment or prevention, provided that the method does not comprise administering tanshinone IIA to a patient suffering from tolerance, sensation or drug addiction.

19. The method of claim 18, wherein said disease or condition is a neurological disease and/or condition.

20. The method of claim 19, wherein said disease or condition is a neurological disease and/or condition associated with the N-receptor.

21. The method of claim 18, wherein said disease or condition is a stroke.

22. The method of claim 21, wherein said stroke is a global ischemic stroke.

23. The method of claim 21, wherein said stroke is a hemorrhagic stroke.

24. The method of claim 21, wherein said stroke is a focal ischemic stroke.

25. The method of claim 18, wherein said disease or condition is chronic pain.

26. The method of claim 18, wherein said disease or condition is acute neurological trauma.

27. The method of claim 18, wherein said disease or condition is general dementia.

28. The method of claim 27, wherein said dementia is Alzheimer's disease.

29. The method of claim 27, wherein said dementia is AIDS-related.

30. The method of claim 27, wherein said dementia is age-related.

31. The method of claim 27, wherein said dementia is multi-infarct.

32. The method of claim 18, wherein said disease or condition is glaucoma.

33. The method of claim 18, wherein said disease or condition is tolerance, sensation and drug addiction provided that said compound is not tanshinone IIA having structure VI.

34. The method of claim 18, wherein said compound is tanshinone.

35. The method of claim 18, wherein said compound is crytotanshinone having structure III.

36. The method of claim 18, wherein said compound is miltirone having structure IV.

37. The method of claim 18, wherein said compound is tanshinone I having structure V.

38. The method of claim 18, wherein said compound is tanshinone IIA having structure VI.

39. The method of claim 18, wherein said compound is tanshinone IIB having structure VII.

40. A method of using a compound having structure I, wherein R₁ and R₂ are independently selected from the group consisting of H, OH and CH₃ and R₃ is selected from the group consisting of H and CH₃ as biological, functional and/or physical markers for establishing safety and standardization protocols for dietary food supplements or complex drugs.

41. The method of claim 40, wherein said compound is tanshinone having structure II.

42. The method of claim 40, wherein said compound is crytotanshinone having structure III.

43. The method of claim 40, wherein said compound is miltirone having structure IV.

44. The method of claim 40, wherein said compound is tanshinone I having structure V.

45. The method of claim 40, wherein said compound is tanshinone IIA having structure VI.

46. The method of claim 40, wherein said compound is tanshinone IIB having structure VII.

47. A method of manufacturing a pharmaceutical composition including the step of extracting any a compound having structure I, wherein R₁ and R₂ are independently selected from the group consisting of H, OH and CH₃ and R₃ is selected from the group consisting of H and CH₃ or utilizing a compound of structure I, wherein R₁ and R₂ are independently selected from the group consisting of H, OH and CH₃ and R₃ is selected from the group consisting of H and CH₃ in its substantially pure form.

48. The method of claim 47, wherein said compound is tanshinone.

49. The method of claim 47, wherein said compound is crytotanshinone having structure III.

50. The method of claim 47, wherein said compound is miltirone having structure IV.

51. The method of claim 47, wherein said compound is tanshinone I having structure V.

52. The method of claim 47, wherein said compound is tanshinone IIA having structure VI.

53. The method of claim 47, wherein said compound is tanshinone IIB having structure VII.
